(19) United States
(12) Patent Application Publication
(10) Pub. No.: US 2004/0137452 A1
(43) Pub. Date: Jul. 15, 2004

(54) Diagnostic test

(76) Inventors: Lisa Julia Levett, London (GB); Stuart Liddle, London (GB)

Correspondence Address: HALE AND DORR, LLP, 60 STATE STREET, BOSTON, MA, 02109

(21) Appl. No.: 10/469,102

(22) PCT Filed: Feb. 26, 2002
(86) PCT No.: PCT/GB02/00839

(30) Foreign Application Priority Data
Feb. 26, 2001 (GB) 0104690.3

(51) Int. Cl. C12Q 1/68

(52) U.S. Cl. 435/6

(57) ABSTRACT

A method for the diagnosis of aneuploidy of a chromosome in a fetus is provided using the polymerase chain reaction. The method utilises a multiplex PCR assay comprising a plurality of chromosome-specific short tandem repeat markers. The method can be used to diagnose fetal trisomy and monosomy responsible for disease conditions such as Down Syndrome and Turner Syndrome, respectively. The method can also be used to diagnose the presence of other genetic conditions such as cystic fibrosis.

The present invention relates to a diagnostic test for the detection of chromosomal abnormalities in a developing fetus and/or a new-born individual, or subsequently during adult growth. The method is based upon analysis of samples using the polymerase chain reaction to quantify fetal DNA.

Chromosomal abnormalities are the most frequently observed genetic disorders in both livebirths and miscarriages. The largest group of chromosomal abnormalities are trisomies, which are reported to lead to 17% of all fetal deaths (Hook, E. B. in Prenatal Diagnosis and Screening, eds. Brock et al, Churchill Livingstone (1992)). Trisomy 21 (Down's Syndrome) has the highest birth prevalence at roughly one affected birth per 700-800 births (Hook, E. B., Obstet. Gynecol. 58 282-285 (1981)).

In the past, traditional karyotyping of cultured amniocytes has proved to be a reliable, cost-effective and accurate means of prenatal genetic diagnosis for a wide range of chromosome abnormalities. However, one disadvantage with the routine test is an extended period during which cells must be cultured prior to analysis. Alternative fluorescent in situ hybridisation-based strategies (FISH) offer the prospect of a more rapid prenatal diagnosis although they add substantially to the laboratory cost of processing amniotic fluid samples (Jalal et al Mayo Clin Proc 73 132-137 (1998); Thilaganathan et al Br J Obstet Gynaecol. 107 262-266 (2000)). Recent studies have examined the use of a polymerase chain reaction (PCR) methodology, on a small number of specimens, and demonstrated its effectiveness for the detection of trisomy 21 (Adinolfi et al Prenat Diagn 17 1299-311 (1997); Toth-et al Prenat Diagn 18 669-74 (1998); Findlay et al J Assist Rep Genet 15 266-75 (1998); Verma et al Lancet 352 9-12 (1998)).

More recent developments have included diagnostic and forensic assays based on the amplification and detection of highly polymorphic classes of repetitive DNA that are present in the human genome for example Boerwinkle et al in Proc. Nat'l Acad. Sci. USA 86 212-216 (1989). The analysis comprises amplification of the DNA segment that contains the repeated sequence by the polymerase chain reaction (PCR). The resulting DNA fragments are then size fractionated by SDS polyacylamide gel electrophoresis (PAGE) or by capillary electrophoresis. Analysis of so-called “short tandem repeat” or STR sequences of DNA have been favoured because of the size and genomic distribution of such sequences, where the repeated unit in such DNA is typically of from 2 to 6 nucleotides. The length of the STR sequence as a whole can be from several tens to several hundreds of nucleotides. An example of this approach in the field of prenatal diagnosis can be found in U.S. Pat. No. 5,994,057.

The application of the PCR technique allows for the detection of chromosomal abnormalities as follows. When primers flanking polymorphic STRs are employed to detect aneuploidies, normal individuals may have either two STR allelic products with a quantitative ratio of 1:1, or could be homozygous with two alleles of the same size. Samples from trisomic patients will either show three different alleles with quantitative dose ratios of 1:1:1 (trisomic tri-allelic), or two PCR products with a ratio of 2:1 (trisomic di-allelic) (Mansfield, E. S. Hum. Mol. Genet. 2 43-50 (1993); Pertl et al Lancet 343 1197-1198 (1994)). It is, though, possible for a trisomic sample to have three similarly sized STR alleles and so represent a single PCR product indistinguishable from a homozygote normal individual. In such circumstances, a diagnosis would not be possible. Efforts to avoid this problem have included the use of a non-polymorphic marker as a control, or several STR markers for each chromosome (Pertl et al Hum. Genet 98 55-59 (1996); Pertl et al Am. J. Obs. Gyn. 177 899-906 (1997)).

However, there are problems with the currently existing methods in terms of reliability and overall accuracy. For example, errors can be introduced through sample contamination in a PCR procedure. Perhaps, the most significant cause of error is allele drop-out (ADO), or the preferential amplification of one allele (Ray et al J. Assist. Reprod. Genet. 13 (2) 104-106 (1996)). This phenomenon can lead to the distortion of the ratio of PCR product obtained. Since the medical decisions made as a result of a positive prenatal diagnosis of aneuploidy have very serious implications, there is a need to provide diagnostic method that is as reliable and as accurate as possible.

It has now been found that such an improvement in reliability and accuracy can be achieved by a method of the present invention, in which the assay of STR markers is undertaken using a co-amplification approach based on at least three simultaneous amplification assays.

According to a first aspect of the present invention there is provided a method for detecting aneuploidy of a chromosome, the method comprising the steps of.

(a) simultaneously amplifying a plurality of chromosome-specific short tandem repeat (STR) markers to form an amplification product mixture comprising copies of the one or more STR markers;

(b) separating the amplified chromosome-specific STR markers from the amplified product mixture according to size;

(c) determining the relative concentrations of the amplified products corresponding to the one or more chromosome-specific STR markers, and

(d) correlating the relative concentration of each amplified STR with the presence or absence of aneuploidy of the chromosome, once consistent results are obtained from at least two markers of the chromosome being assayed.

wherein at least three simultaneous assays are carried out according to steps (a) to (c) prior to step (d).

The condition of aneuploidy (or heteroploidy) refers to the condition of a cell nucleus having more than or less than an integral multiple of the typical haploid chromosome number. The term includes the conditions of monosomy where one chromosome of a chromosome pair is missing and trisomy where an additional copy is present. In some rare cases it is also possible for an individual to have two or more extra chromosomes.

The normal diploid number of chromosomes in humans is 46. Individuals with chromosome counts that are not multiples of the normal haploid number (23) are said to be aneuploid. A fetus can receive higher multiples of the haploid number of chromosomes to give 69 (3-times) or 92 (four-times) chromosomes. Such triploid or tetraploid fetuses normally miscarry early during pregnancy.

Methods of the present invention are generally applicable to diagnosis of aneuploidy in any animal species. In general, though, it is with respect to human medicine that such methods are expected to have the greatest applicability. The present invention is not limited, though, in this respect and extends to such methods carried out on samples from any animal species, in particular mammalian species, for example, primates (including apes and monkeys), and ungulates (including bovine, ovine, caprine, porcine, canine, feline and equine species). The methods can, of course, be used in the analysis of the cellular material from transgenic or cloned animals, in particular transgenic or cloned non-human animals, preferably non-human mammals.

As noted above, methods of the present invention have particular importance with regard to the diagnosis of aneuploidy in a fetus. Although, it should be noted that the methods are applicable to the determination of the chromosomal complement in any cell, i.e. all somatic and germ cells in an individual. In terms of a developing fetus, the methods may be practised on any cell, so from the one-cell zygote stage, through, the various embryonic stages to the development of the fetus. The methods can also be practised on any cell from the new born individual into subsequent growth and development. Additionally, there are sources of fetal DNA present in maternal plasma. In these samples, the DNA is free from the cell.

Examples of human disease conditions caused by aneuploidy include, but are not limited to, Down Syndrome (trisomy 21) i.e. three copies of chromosome 21, Edwards Syndrome (trisomy 18), Patau Syndrome (trisomy 13), Turner Syndrome (monosomy X) i.e. only one X chromosome in females, Kleinfelter Syndrome (XXY) in males, Triple X Syndrome (XXX) and other conditions such as (XYY).

The number of STR markers (STR regions) assayed according to a method of the present invention, comprises a plurality of STR markers, preferably at least two, three or four, or at least five or six, or at least six or seven STR markers. Each STR marker being independently amplified in each of the at least three assays. Additional STR markers can be considered and independently included, so the number can be six, seven, eight, nine or ten, or more independently in total in each separate assay. Each assay can therefore contain a different number of markers.

In general, the STR marker DNA to be analysed is amplified by the polymerase chain reaction (PCR), a technique which is now standard in molecular biology laboratories (see for example, U.S. Pat. No. 4,683,195; U.S. Pat. No. 4,800,159; U.S. Pat. No. 4,965,188; U.S. Pat. No. 4,683,202; U.S. Pat. No. 4,889,818; and Innis et al, Editors, PCR Protocols (Academic Press, New York, 1990). Simultaneous co-amplification is sometimes referred to as multiplex PCR assay.

Primers for PCR amplification may be readily synthesised by standard techniques, for example by solid phase synthesis via phosphoramidite chemistry (U.S. Pat. No. 4,458,066; U.S. Pat. No. 4,415,732; Beaucage et al, Tetrahedron, 48, 2223-2311 (1992)).

Chromosome-specific STR markers can be selected by choosing synthesising primers that hybridise to adjacent unique sequence regions. The unique sequence regions will ensure that only the STR specific for the desired chromosome will be amplified. Appropriate STRs may be identified from publicly available DNA sequence data bases, such as GeneBank™ or can be identified from libraries of chromosome-specific DNA libraries using the method described by Edwards et al, Am. Hum. Genet 49 746-756 (1991). STR markers can be obtained from the genome database (www.gdb.org) or the publication of the human genome (Science 291 1304-1351 (2001); Nature 409 813-958 (2001)) can be inspected. The STR marker can be selected from any desired locus on a chromosome that has the necessary heterozygosity. For disease conditions known to be associated with one of chromosomes 13, 18, 21, X or Y, the STR marker can be selected from a loci on the chromosome as appropriate.

Several factors can affect the selection of primers for amplification, for example the relative stability of the primers when bound to target DNA-which largely depends on relative GC content, the presence or absence of secondary structures in the target DNA, relative length of primers (Rychlik et al, Nucleic Acids Research, 17 8543-8551 (1989); Lowe et al, Nucleic Acids Research, 18 1757-1761 (1990); Hillier et al, PCR Methods and Applications, 1 124-128 (1991)). Where a PCR machine is used, the STRs can be amplified by 20 to 35 PCR cycles, suitably by 25 to 30 PCR cycles.

As used herein, the term “PCR primers” refers to primer complementary to sequences adjacent to an STR to be amplified. The PCR primers may be suitably in the range of from 15 to 35 nucleotides long, or in the range of from 10 to 50 nucleotides long, up to about 100 to 400 nucleotides of the STR to be amplified.

In methods of the present invention, it may be preferable for the amplification products (i.e. the copies of STR DNA produced in the amplification step) to be labelled to facilitate their quantification after separation. A variety of different labelling approaches are suitable for use with the present invention, including the direct or indirect attachment of radioactive labels, fluorescent labels, electron dense labels. There are several means available for derivatising oligonucleotides with reactive functional groups which permit the addition of a label. For example, several approaches are available for biotinylating a PCR primer so that fluorescent, enzymatic, or electron density labels can be attached via avidin (Broken et al, Nucleic Acids Research 5 363-384 (1978)), or by biotinylation of the 5′ termini of oligonucleotides via an aminoalkylphosphoramide linker arm (Chollet et al, Nucleic Acids Research 13 1529-1541 (1985)). Several methods are also available for synthesising amino-derivatised oligonucleotides which are readily labelled by fluorescent or other types of compounds derivatised by amino-reactive groups, such isothiocyanate, N-hydroxysuccinimide, (Connolly, Nucleic Acids Research 15.3131-3139 (1987); Gibson et al, Nucleic Acids Research 15 6455-6467 (1987); U.S. Pat. No. 4,605,735). Methods are also available for synthesising sulfhydryl-derivatised oligonucleotides which can be reacted with thiol-specific labels, (U.S. Pat. No. 4,757,141; Connolly, Nucleic Acids Research 13.4485-4502 (1985); Spoat et al, Nucleic Acids Research, 15 4837-4848 10(1987). A comprehensive review of methodologies for labelling DNA fragments is provided by Matthews et al, Anal. Biochem., 169 1-25 (1988).

Amplified STR DNA can be labelled fluorescently by linking a fluorescent molecule to one or more primers (U.S. Pat. No. 4,757,141; U.S. Pat. No. 4,855,225). Preferably, copies of different STRs are labelled with different fluorescent labels to facilitate quantitation. (Smith et al, Methods in Enzymology 155 260-301 (1987); Karger et al., Nucleic Acids Research 19 4955-4962 (1991); Haugland, Handbook of Fluorescent Probes and Research Chemicals (Molecular Probes, Inc., Eugene, 1989). Preferred fluorescent labels include, fluorescein and derivatives thereof (U.S. Pat. No. 4,318,846; Lee et al, Cytometry 10 151-164 (1989)), tetramethylrhodamine, rhodamine X, Texas Red, and other related compounds. Most preferably, when a plurality of fluorescent dyes are employed they are spectrally resolvable, as taught by Fung et al (cited above). Briefly, as used herein “spectrally resolvable” fluorescent dyes are those with quantum yields, emission bandwidths, and emission maxima that permit electrophoretically separated polynucleotides labelled thereby to be readily detected despite substantial overlap of the concentration bands of the separated polynucleotides.

PCR primers of the invention can also be radioactively labelled with phosphorous-32 using standard protocols, e.g. Maniatis et al, in Molecular Cloning: A Laboratory Manual (Cold Spring Harbor Laboratory, New York, (1982)); Current Protocols in Molecular Biology, Unit 6.4 (John Wiley & Sons, New York, (1987)); or Maxim and Gilbert, Meth. Enzymol., 65 499-560 (1980).

Separation of the amplified STRs from a sample by size fractionation may be accomplished in a variety of ways, including by filtration, high performance liquid chromatography, electrophoresis, affinity collection (Syvanen et al, Nucleic Acids Research, 16 11327-11338 (1988)). The amplified STRs can be separated from the amplified product mixture by gel electrophoresis or capillary electrophoresis. Alternatively, the amplified STRs can be fluorescently labelled and separated by gel electrophoresis or capillary electrophoresis (Mayrand et al, Clinical Chemistry 36 2063-2071 (1990); Mayrand et al, Annales de Biologie Clinique 91 224-230; Mayrand et al, Appl. and Theoret. Electrophoresis 3 1-11 (1992)).

Chromosomal DNA of an individual who is being tested for aneuploidy is obtained from a cell sample from that individual or from a cell-free source, such as maternal blood plasma (Prenatal Diagnosis 20 795-798 (2000)). Cell samples can be obtained from a variety of tissues depending on the age and condition of the individual. Samples (cell or cell-free) may be obtained from peripheral blood using standard techniques. Preferably, in fetal testing, a sample is obtained by amniocentesis, chorionic villi sampling, or a sample of maternal blood plasma. Preferably, DNA is extracted from the sample using standard procedures, e.g. phenol:chloroform extraction as described by Maniatis et al, in Molecular Cloning (Cold Spring Harbor, N.Y., (1982)). Cell samples for fetal testing can also be obtained from maternal peripheral blood using fluorescence-activated cell sorting (Iverson et al, Prenatal Diagnosis, 9.31-48 (1981)).

The correlation of the relative concentration of each amplified STR marker with the presence or absence of aneuploidy can be undertaken in any generally convenient means. The ratios of amplified STR marker products obtained in the method are analysed and the diagnosis of the condition of the chromosomes in the sample can be made.. Consistent results from at least two markers of the chromosome being assayed (with no opposing results) are required for an accurate diagnosis according to a method of the present invention.

In practice, diagnosis is only accurate when markers are selected carefully which produce smaller variation between allele size to avoid preferential amplification but maintain a high (over 70%) heterozygosity. A preferred embodiment of the present invention, includes a method as described above in which the STR markers have a high heterozygosity, of at least 70%, up to 75%, 80%, 85%, 90%, 95% or 100%.

In certain circumstances it may be convenient to arrange for an additional amplification assay to be carried out. For example, where a particular selection of markers has not yielded a clear result and only one SIR marker shows a double-peak indicative of heterozygosity, then a further assay of additional STR markers can be used to confirm a diagnosis. Such additional markers can be used in combinations, for example up to 3, or more, to provide the additional data.

The STR markers that can be used in accordance with methods of the present invention, include but are not limited to:

D13S257, DXS981, D13S631, D21S1414, DXS6785, D18S536, D13S258, D18S535, D21S1411, D18S548, 21-32S (informal designation), D21S11, DXS7423, HPRT (or XHPRT), D21S1446, D18S391, X-61 (informal designation), D18S978, D13S317, D13S627, D13S800 D18S1002.

In such multiplex PCR methods other STR markers for the simultaneous detection of aneuploidy and markers for other gene defects can be used, such as for example, the marker indicative of the presence or absence of the cystic fibrosis ΔF508 mutation, CF508. Other markers include, but are not limited to, HbS for Sickle Cell Anaemia and IVS1-110 for β-thalassaemia (Sherlock et al Ann. Hum. Genet. 62 9-23 (1998)), and RHO for Rhesus status (Zhong et al Br. J. Obstet Gynaecol. 107 766-767 (2000)).

Quantitative markers such as AMEL A and AMEL B may also be included in the multiplex assays to detect anomalies arising mainly from second meiotic division non-disjunction. The markers can be jointly referred to as “AMEL A+B”.

The DNA to be analysed can be obtained from any generally suitable cell, fluid or tissue source. In prenatal diagnosis, cells may be obtained from the developing fetus directly by tissue biopsy, or a sample of amniotic fluid or following chorionic villus sampling. In new born babies, children or adults, the sample can be obtained from any convenient tissue source, including, for example, blood or buccal swabs.

The results of the amplification procedure may be analysed using a DNA sequencer. For example, DNA sequencer apparatus supplied by Applied Biosystems. The relative amounts of amplification product can be quantitated according to the label used, e.g. fluorescent dye or radioactive label. When the results are displayed graphically, the area under the peak on the output from the sequence analyser can be used to quantitate the amount of amplification product present for each DNA marker.

In reaching a diagnosis of trisomy, the ratios of the peaks obtained for each amplification product are compared. Methods in accordance with the present invention permit a diagnosis of a normal chromosomal complement with a peak ratio in the range 1:1 to 1.4:1 for a particular STR marker. A diagnosis of di-allelic trisomy (diplozygous trisomy) can be made when the peak ratio is above 1.6:1. The identification of these ratio values is important as false negative results are avoided.

In a preferred embodiment of the invention, there is provided a method as described in accordance with the first aspect of the invention, in which the at least three simultaneous assays (or mulitplex mixes) each comprise independently at least six different STR markers (at least two markers for each chromosome being assayed for), and in which a peak value ratio of amplification product of a STR marker of 1:1 to 1.4:1 is diagnostic of a normal complement of chromosomes and a peak value ratio of 1.6:1 or above is diagnostic of di-allelic trisomy.

According to a second aspect of the present invention, there is provided a kit of parts comprising at least three multiplexes of labelled primers for carrying out a method of the present invention as described above. Suitably such kits can include at least 3 sets of labelled primers for the STR markers to be amplified, polymerase buffer solution in which a DNA polymerase can extend the primers in the presence of DNA polymerase, and deoxynucleoside triphosphates. The labelled primers may include fluorescent labels and the DNA polymerase may be Taq DNA polymerase. The fluorescent labels include, but are not limited to, fluorescein, rhodamine, and derivatives thereof, including carboxyfluorescein, 4,7-dichlorofluoresceins, tetramethylrhodamine, rhodamine X, or derivatives thereof.

According to a third aspect of the invention there is provided the use of STR marker 21-32S (informal designation) as a marker for the diagnosis of aneuploidy of a chromosome. The method may be as described above in relation to the first aspect of the invention, or alternatively, the method may be any generally suitable diagnostic test. For example the method described in U.S. Pat. No. 5,994,057, Pertl et al in Am. J. Obstet. Gynecol. 177 (4) 899-906 (1997), or Verma et al in Lancet 352 9-12 (1998).

According to a fourth aspect of the invention there is provided the use of the marker Y-40S (informal designation) as a marker for the diagnosis of the sex of an individual. The method may be as described above in relation to the first aspect of the invention, or alternatively, the method may be any generally suitable diagnostic test. For example the method described in U.S. Pat. No. 5,994,057, Pertl et al in Am. J. Obstet. Gynecol. 177 (4) 899-906 (1997), or Verma et al in Lancet 352 9-12 (1998). In most cases, such methods will be of greatest use in detecting the sex of an unborn fetus.

According to a fourth aspect of the invention there is provided the use of marker CF508 (informal designation) as a marker for the diagnosis of the most frequent cystic fibrosis mutation in the DNA of an individual. The method may be as described above in relation to the first aspect of the invention, or alternatively, the method may be any generally suitable diagnostic test. For example the method described in U.S. Pat. No. 5,994,057, Pertl et al in Am. J. Obstet. Gynecol. 177 (4) 899-906 (1997), or Verma et al in Lancet 352 9-12 (1998).

Unlike FISH, methods of the present invention are feasible on very small volumes of amniotic fluid (0.3 to 1 ml), which does not then compromise any cell culture requirements. The PCR methodology amplifies DNA from cells and therefore does not rely on the cells being alive or intact. This allows the technique to be used on samples taken at both earlier (12 weeks) or later gestations (34 weeks), when samples are lacking an abundance of live cells, without affecting its reliability. Amnio-PCR can be easily scaled up to cope with large numbers of samples (240 samples per 24 hours per 3700 ABI DNA Sequencer).

Methods according to the present invention may also be used for amnio-PCR analysis for comparative DNA studies, for example to determine the zygosity studies of twins and for paternity determination.

Concerns about maternal contamination in cell cultures have been alleviated simply by comparing the DNA profiles from the maternal DNA obtained from a mouthwash sample to the profile from the amniotic fluid DNA.

In a preferred embodiment of the invention, there is provided a method for detecting aneuploidy of a chromosome comprising the following steps:

(1) preparing sample(s) of amniotic fluid for analysis;

(2) selecting appropriate chromosome-specific short tandem repeat (STR) markers for use in at least 3 simultaneous multiplex reactions;

(a) including preparation of negative control with no template DNA;

(3) labelling the forward or reverse primer from each pair of markers used with appropriate label (e.g. fluorescent dye, radioactive label);

(4) preparing sample mixtures for polymerase chain reaction (PCR);

(5) amplification of DNA sequences in samples using PCR;

(6) separation of amplified DNA samples, e.g. by electrophoresis;

(7) quantification of DNA representing each allele amplified for a specific marker used in the 3 simultaneous multiplex reactions;

(8) analysis of ratios of peak area for each allele amplified and determination of chromosome status of fetus

The samples for analysis may be frozen, or if routine culture is to be performed in addition then samples are at room temperature.

The extraction of DNA from the cells may be performed by any convenient means. The cells may be resuspended and a 1.0 ml aliquot centrifuged in a microfuge tube. The pellet of cells may then be resuspended in a suitable medium such as phosphate buffered saline to wash the cells. The pellet may then be resuspended with Chelex™ resin and incubated at an appropriate temperature of at least 50° C., preferably 56° C. and no more than 60° C. The DNA thus obtained is denatured by heating at 100° C. and then centrifuged.

The multiplex PCR may be performed as follows. For each sample or control, three sets of tubes are prepared each containing one of the three different multiplex mixes of probe/primer sets as desired. Supernatant containing DNA from the cell sample is then pipetted into each set of tubes, including controls. The sample tubes thus prepared are subjected to PCR using a convenient apparatus.

At the end of the PCR, samples are separated be gel or capillary electrophoresis using conventional fluorescent DNA analysers. Identification and quantification of DNA product can be performed using any convenient method., e.g. ABI GeneScan™. The DNA fragment size, chromosomal origin and quantification can then be determined using any generally convenient means, for example an ABI Genotyper™. Markers are identified for each chromosome pair and are classified by comparison to results from known samples.

In such a method, markers producing three peaks with an approximate peak area ratio of 1:1:1 (i.e. below 1.4) are considered consistent with trisomy. Heterozygous markers producing two peaks with a DNA ratio below 1.4 are considered to be consistent with euploidy and a ratio above 1.6 consistent with trisomy. Any ratio between 1.4 and 1.6 is considered to be inconclusive. PCR reactions producing inconclusive ratios may be repeated to clarify the result. In cases where only one marker from a single chromosome is found to be heterozygous, an extra multiplex system comprised of at least two different DNA markers per chromosome can be used. Positive and consistent results from at least two informative markers for each chromosome are required before a conclusion is drawn.

Preferred features for the second and subsequent aspects of the invention are as for the first aspect mutatis mutandis.

In the specification, reference is made to a number of drawings in which

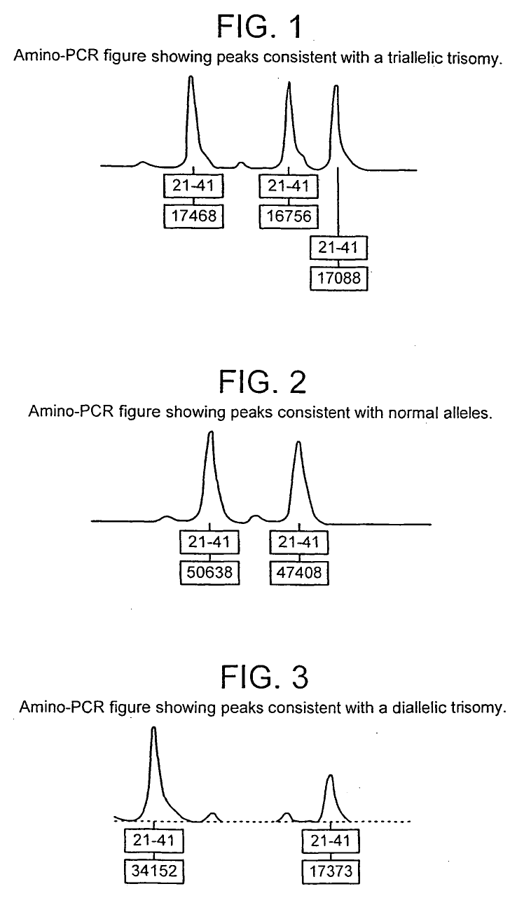

FIG. 1 shows ammo-PCR results from a sample consistent with a tri-allelic trisomy. The code “21-41” indicates the marker D21S1414.

FIG. 2 shows amnio-PCR results from a sample consistent with normal alleles. The code “21-41” indicates the marker D21S1414

FIG. 3 shows amnio-PCR results consistent with a di-allelic trisomy. The code “21-41” indicates the marker D21S1414

The present invention will now be further described with reference to the following Examples, which are included for the purposes of illustration and are not to be construed as being limiting on the invention.

### EXAMPLE 1

### Detection of Trisomies 21, 18 and 13, and Sex Chromosome Anomalies

Amnio-PCR is the amplification and quantification of specific genomic DNA regions from uncultured amniocytes using PCR methodology. The following examples show the reliability and accuracy of Amnio-PCR for the rapid prenatal diagnosis of genetic abnormality, specifically trisomy 21, 18, 13 and sex chromosome anomalies.

Methods

This was a prospective study conducted between March 1999 and March 2000. Amniocentesis was performed on 5097 women from a number of specialist prenatal diagnostic units. Procedures were performed between 12 and 34 weeks gestation for a number of indications including advanced maternal age, positive biochemical screening for Down syndrome, fetal anomalies detected on ultrasound examination, positive maternal viral titres or maternal anxiety.

Upon receipt at the laboratory, amniotic fluid samples were divided into three aliquots. 1 ml of amniotic fluid was used for Amnio-PCR and the remainder used to establish two simultaneous cultures for the conventional cytogenetic analysis of metaphase chromosomes. All macroscopically bloodstained amniotic fluid specimens were excluded from the study and these samples accounted for approximately 2% (n=97) of clinical procedures. DNA was extracted from uncultured amniocyte samples, using a ReadyAmp DNA extraction kit (Promega UYK Ltd).

Twenty-four tetranucleotide repeat markers obtained from the genome database (www.gdb.org) were amplified by PCR in three multiplexes. Six DNA markers were used for each of the autosomes 18 and 13, together with five markers for each of chromosome 21 and the X-chromosome and two for the Y-chromosome. Negative controls with no template DNA were included for each of the multiplexes on every run.

In each case the forward primer from each pair was labelled with a fluorescent dye. Primers were included at varying concentrations to ensure comparable amounts of PCR products. The final multiplex mixes consist of the relevant primer sets, 1× Amplitaq Gold buffer with 1.5 mM MgCl₂ (Applied Biosystems, USA), 0.2 mM dNTPs (Promega, USA) and 0.25 units Amplitaq Gold DNA Polymerase (Applied Biosystems, USA) per reaction. Reactions are performed in a final volume of 10 μl with 1 μl of extracted DNA. All three multiplexes are amplified under the following PCR conditions: 94° C. for 15 minutes for 1 cycle, 93° C. for 48 seconds, 60° C. for 48 seconds and 72° C. for 1 minute for 35 cycles. A final extension is added at 72° C. for 5 minutes. The three multiplexes used were as follows:

Multiplex 1

| | Concentration (pMol per | |
|---|---|---|
| Primer name | 10 μl reaction) | Reference |
| D13S257 | 4.3 | GDB ID: G00-198-826 |
| DXS981 | 2.4 | GDB ID: G00-187-674 |
| D13S631 | 1.6 | GDB ID: G00-315-353 |
| D21S1414 | 1.8 | GDB ID: G00-315-821 |
| DXS6785 | 1.0 | GDB ID: G00-198-440 |
| D18S536 | 0.9 | GDB ID: G00-686-892 |
| D13S258 | 1.3 | GDB ID: G00-198-849 |
| AMELA + B | 0.6 | Biotechniques 15 636-641 |
| | | (1993) |

Multiplex 2

| | Concentration (pMol per | |
|---|---|---|
| Primer name | 10 μl reaction) | Reference |
| D18S535 | 1.5 | GDB ID: G00-868-499 |
| D21S1411 | 3.7 | GDB ID: G00-313-723 |
| D18S548 | 2.8 | GDB ID: G00-686-697 |
| 21-32S | 3.4 | Sequence Below |
| Y-40S | 1.0 | Sequence Below |
| D21S11 | 1.6 | GDB ID: G00-188-664 |
| DXS7423 | 0.5 | GDB ID: G00-574-202 |
| HPRT | 0.6 | GDB ID: G00-181-411 |
| CF508 | 0.6 | Sequence Below |

Multiplex 3

| | Concentration (pMol per | |
|---|---|---|
| Primer name | 10 μl reaction) | Reference |
| D21S1446 | 1.6 | GDB ID: G00-685-533 |
| D18S391 | 0.9 | GDB ID: G00-198-725 |
| X-61 | 1.0 | Sequence Below |
| D18S978 | 0.6 | GDB ID: G00-685-899 |
| D13S317 | 0.8 | GDB ID: G00-686-856 |
| D13S627 | 0.6 | GDB ID: G00-314-629 |
| D13S800 | 0.7 | GDB ID: G00-685-362 |

The sequences of novel primers 21-32S, Y-40S, X-61 and CF508 are as follows:

```
Primer 32S:
              Reverse
              GGG AAG GCT ATG GAG GAG A
              Forward
              CTC CAG CCT GGG TGA CAA G
Primer 40S:
              Reverse
              GCA TCT TCG CCT TCC GAC GAG
              Forward
              GAA TAT TCC CGC TCT CCG GA
Primer X-61:
              Reverse
              AGA GGA GTT GCA ACC CAG A
              Forward
              ATT GAA GAA GGC ACC TTT CAG C
Primer CF508:
              Reverse
              TTC TAG TTG GCA TGC TTT GAT GAC GCT TC
              Forward
              AGT TTT CCT GGA TTA TGC CTG GCA C
```

The amplified DNA samples were separated by electrophoresis on an ABI 377 DNA sequencer (Applied Biosystems, Forster City, US), and the DNA representing each allele for a specific marker was quantified by its peak area using Genotyper 2.5 software (Applied Biosystems). The peak area ratio between each allele was calculated. Markers producing three peaks with an approximate peak area ratio of 1:1:1 (i.e. below 1.4) were considered consistent with trisomy, as shown in FIG. 1. Heterozygous markers producing two peaks with a DNA ratio below 1.4 were considered to be consistent with euploidy, shown in FIG. 2, and a ratio above 1.6 consistent with trisomy, shown in FIG. 3. Any ratio between 1.4 and 1.6 was considered to be inconclusive. PCR reactions producing inconclusive ratios were repeated to clarify the result. In rare cases where only one marker from a single chromosome was found to be heterozygous, an extra multiplex system comprised of two different DNA markers per chromosome was used. Positive and consistent results from at least two informative markers for each chromosome were required before a conclusion could be drawn.

Results

Amnio-PCR results were available in an average of 2 days from receipt of specimen. The sample failure rate was 0.1%: five samples failed to give a clear result for any of the chromosomes, one due to gross fungal infection and four due to excessive contamination with solid maternal tissue. The maternally contaminated samples showed more than two peaks with differing peak areas at most of the loci. These cases could be distinguished from triploid or trisomy cases as loci showing three peaks had peak area ratios of 1:2:1 within the defined parameters according to this study. Other loci showed large peaks, representing fetal DNA and much smaller peaks representing maternal DNA; the latter was confirmed by comparing DNA from a mouthwash sample from the mother with the DNA from the amniotic fluid sample. The ratios between these peaks varied depending on the extent of contaminating maternal DNA

Of the 4,995 samples successfully processed, 89 autosomal anomalies were identified by Amnio-PCR, as shown in Table 1, all subsequently verified by conventional karyotyping. There were no false positive or false negative results amongst the autosomal loci evaluated. Sixteen sex chromosomes aberrations were also detected, shown in Table 1, with subsequent chromosome analysis confirming these diagnoses and identifying four additional sex chromosome anomalies (one 47,XYY and three 47,XXY). Failure of PCR in these cases was due to an absence of marker heterozygosity in the sex chromosome regions labelled. Initial screening for sex chromosome anomalies was found to be accurate for cases involving first meiotic division non-disjunction whilst specific quantitative markers (now included in multiplex as “AMEL A+B”) were necessary to detect those anomalies arising mainly from second meiotic division non-disjunction (Sullivan et al BioTechniques 15 636-41 (1993); Cirigliano et al Prenat Diagn 19 1099-103 (1999)).

TABLE 1
Number of chromosomal abnormalities detected by conventional
cytogenetic and Amnio-PCR analysis.

| | Amnio-PCR | Cytogenetically |
|---|---|---|
| Condition | Positive | Abnormal |
| Trisomy 21 | 57 | 57 |
| Trisomy 18 | 17 | 17 |
| Trisomy 13 | 8 | 8 |
| Triploidy | 7 | 7 |
| Sex chromosome | 16 | 20 |
| Anomalies | | |
| Mosaics | 0 | 16 |
| Balanced structural | 0 | 17 |
| Anomalies | | |
| Unbalanced structural | 0 | 12 |
| Anomalies | | |
| Total | 105 | 154 |

In 2% of cases (n=94), amnio-PCR analysis remained inconclusive for one of the marked chromosomes alone, even after using the extra markers. Such assays included those where 7 out of 8 markers for one particular chromosome showed a homozygous peak (the remaining marker was normal in all cases) and other assays which consistently gave ratios between 1.4 and 1.6 even when repeated. In these cases, a report was issued for the chromosomes with conclusive results and a culture analysis subsequently clarified the result for the remaining chromosome.

The sensitivity of Amnio-PCR for the detection of all chromosomal abnormalities was 68.2% (105/154). The false-negative rate was 3.7% (4/109) and the false positive rate was 0% (0/154). The detection rate for trisomies 21, 18, 13 and triploidy was 100% (89/89), as shown in Table 2.

TABLE 2
The detection capabilities of Amino-PCR compared to conventional
karyotyping

| | | Detected by | Not detected by |
|---|---|---|---|
| Condition | Total | Amnio-PCR | Amnio-PCR |
| All chromosomal | 154 | 105 (68.2%) | 49 (31.8%) |
| Abnormalities | | | |
| Chromosomal abnormalities | 109 | 105 (96.3%) | 4 (3.7%) |
| Potentially detectable by | | | |
| Amnio-PCR | | | |
| Detection of trisomies 21, 18 and | 89 | 89 (100%) | 0 (0%) |
| 13 and triploidy | | | |

Discussion

The findings of this study demonstrate that Amnio-PCR is a fast and reliable technique for the diagnosis of most major chromosomal abnormalities. As Amnio-PCR was diagnostic for the autosomal trisomies, the commonest abnormalities routinely screened for antenatally, this technique is likely to alleviate parental stress and anxiety that is so often felt during the wait for a culture result.

Amnio-PCR prospectively identified 68% of all chromosome anomalies diagnosed by conventional cytogenetic analysis, however, approximately 85% of balanced structural chromosome abnormalities—which were not detected by amnio-PCR—result in normal birth outcome. Therefore, when only karyotypic abnormalities that result in an abnormal pregnancy outcome are considered, the detection rate increases to 75%, as shown in Table 2. Mosaics, translocations and deletion/duplication syndromes are not likely to be detected by the Amnio-PCR technique described. This will limit the potential of the Amnio-PCR in its present form, to completely replace conventional cytogenetic analysis.

Amnio-PCR is an accurate and reliable technique for the prenatal diagnosis for major chromosomal abnormalities—trisomies 21, 18 and 13 and the sex chromosome anomalies. It is shown here to be especially reliable for the autosomal trisomies where the detection rate in this study is 100%. The speed of the methodology will help to minimise the period of parental anxiety in the wait for a diagnostic test result. Amnio-PCR is likely to become an essential adjunct to traditional cytogenetic analysis.

### EXAMPLE 2

### Detection of Trisomies 21, 18 and 13, and Sex Chromosome Anomalies

A variant of the PCR multiplex described in Example 1 was performed as previously described. The three multiplexes used were as follows:

Multiplex 1

| | Concentration (pMol per | |
|---|---|---|
| Primer name | 10 μl reaction) | Reference |
| D13S257 | 4.3 | GDB ID: G00-198-826 |
| DXS981 | 2.4 | GDB ID: G00-187-674 |
| D13S631 | 1.6 | GDB ID: G00-315-353 |
| D21S1414 | 1.8 | GDB ID: G00-315-821 |
| DXS6785 | 1.0 | GDB ID: G00-198-440 |
| D18S536 | 0.9 | GDB ID: G00-686-892 |
| D13S258 | 1.3 | GDB ID: G00-198-849 |
| AMELA + B | 0.6 | Biotechniques 15 636-641 |
| | | (1993) |
| D18S1002 | 2.0 | GDB ID: G00-555-855 |

Multiplex 2

| | Concentration (pMol per | |
|---|---|---|
| Primer name | 10 μl reaction) | Reference |
| D18S535 | 1.5 | GDB ID: G00-868-499 |
| D21S1411 | 3.7 | GDB ID: G00-313-723 |
| D18S548 | 2.8 | GDB ID: G00-686-697 |
| 21-32S | 3.4 | Sequence Below |
| Y-40S | 1.0 | Sequence Below |
| D21S11 | 1.6 | GDB ID: G00-188-664 |
| DXS7423 | 0.5 | GDB ID: G00-574-202 |
| XHPRT | 0.6 | GDB ID: G00-181-411 |
| CF508 | 0.6 | Sequence Below |

Multiplex 3

| | Concentration (pMol per | |
|---|---|---|
| Primer name | 10 μl reaction) | Reference |
| D21S1446 | 1.6 | GDB ID: G00-685-533 |
| D18S391 | 0.9 | GDB ID: G00-198-725 |
| X-61 | 1.0 | Sequence Below |
| D18S978 | 0.6 | GDB ID: G00-685-899 |
| D13S317 | 0.8 | GDB ID: G00-686-856 |
| D13S627 | 0.6 | GDB ID: G00-314-629 |
| D13S800 | 0.7 | GDB ID: G00-685-362 |

The sequences of novel primers 21-32S, Y-40S, X-61 and CF508 are as given above.

The results from 3 separate multiplex PCR analyses on 3 individual samples using these multiplex markers, showing the assessment of chromosome and sex status of the fetus, were as follows:

Multiplex 1

| | | | | | |
|---|---|---|---|---|---|
| Primer | Peak (i) | Peak (ii) | Area ratio | Labels | Diagnosis |

Sample 1

| | | | | | |
|---|---|---|---|---|---|
| D13S257 | 24700 | 22420 | 1.102 | 2 | Normal |
| D13S631 | 34317 | (—) | (—) | 1 | Homozygous |
| | | | | | marker |
| D13S258 | 58852 | 53853 | 1.093 | 2 | Normal |
| D18S536 | 85875 | 77844 | 1.103 | 2 | Normal |
| D18S1002 | 21676 | 22609 | 1.043 | 2 | Normal |
| D21S1414 | 19921 | 16279 | 1.224 | 2 | Normal |
| AMELA + B | 67041 | (—) | (—) | 1 | Female |
| DXS981 | 11962 | 11344 | 1.054 | 2 | Disomic X |
| DXS6785 | 71718 | (—) | (—) | 1 | Homozygous |
| | | | | | marker |

Sample 2

| | | | | | |
|---|---|---|---|---|---|
| D13S257 | 25113 | 22619 | 1.110 | 2 | Normal |
| D13S631 | 18733 | 24137 | 1.288 | 2 | Normal |
| D13S258 | 60764 | 39327 | 1.545 | 2 | Inconclusive |
| D18S536 | 56236 | 47371 | 1.187 | 2 | Normal |
| D18S1002 | 74389 | (—) | (—) | 1 | Homozygous |
| | | | | | marker |
| D21S1414 | 29713 | 22580 | 1.316 | 2 | Normal |
| AMELA + B | 23385 | 17874 | 1.308 | 2 | Male |
| DXS981 | 15227 | (—) | (—) | 1 | Homozygous |
| | | | | | marker |
| DXS6785 | 59597 | (—) | (—) | 1 | Homozygous |
| | | | | | marker |

Sample 3

| | | | | | |
|---|---|---|---|---|---|
| D13S257 | 34837 | 30445 | 1.144 | 2 | Normal |
| D13S631 | 26896 | 23698 | 1.135 | 2 | Normal |
| D13S258 | 114832 | (—) | (—) | 1 | Homozygous |
| | | | | | marker |
| D18S536 | 103426 | 82328 | 1.256 | 2 | Normal |
| D18S1002 | 45681 | 34862 | 1.310 | 2 | Normal |
| D21S1414 | 34205 | 31404 | 1.089 | 2 | Normal |
| AMELA + B | 47408 | 36971 | 1.282 | 2 | Male |
| DXS981 | 19598 | (—) | (—) | 1 | Homozygous |
| | | | | | marker |
| DXS6785 | 57319 | (—) | (—) | 1 | Homozygous |
| | | | | | marker |

Multiplex 2

| | | | | | |
|---|---|---|---|---|---|
| Primer | Peak (i) | Peak (ii) | Area ratio | Labels | Diagnosis |

Sample 1

| | | | | | |
|---|---|---|---|---|---|
| D18S535 | 71439 | 63858 | 1.119 | 2 | Normal |
| D18S548 | 9259 | 8069 | 1.147 | 2 | Normal |
| D21S11 | 24941 | 20783 | 1.200 | 2 | Normal |
| 21-32s | 9229 | 6679 | 1.382 | 2 | Normal |
| D21S1411 | 28566 | 21745 | 1.314 | 2 | Normal |
| CF*508 | 74854 | (—) | (—) | 1 | Normal |
| CF*508del | (—) | (—) | (—) | 0 | |
| Y-40s | (—) | (—) | (—) | 0 | |
| XHPRT | 80860 | (—) | (—) | 1 | Homozygous |
| | | | | | marker |
| DXS7423 | 55980 | (—) | (—) | 1 | Homozygous |
| | | | | | marker |

Sample 2

| | | | | | |
|---|---|---|---|---|---|
| D18S535 | 109450 | (—) | (—) | 1 | Homozygous |
| | | | | | marker |
| D18S548 | 7615 | 6610 | 1.152 | 2 | Normal |
| D21S11 | 17061 | 14103 | 1.210 | 2 | Normal |
| 21-32s | 20238 | 15250 | 1.327 | 2 | Normal |
| D21S1411 | 42720 | 47765 | 1.118 | 2 | Normal |
| CF*508 | 28384 | (—) | (—) | 1 | Normal |
| CF*508del | (—) | (—) | (—) | 0 | |
| Y-40s | 6730 | (—) | (—) | 1 | Male |
| XHPRT | 38263 | (—) | (—) | 1 | Homozygous |
| | | | | | marker |
| DXS7423 | 40934 | (—) | (—) | 1 | Homozygous |
| | | | | | marker |

Sample 3

| | | | | | |
|---|---|---|---|---|---|
| D18S535 | 138068 | (—) | (—) | 1 | Homozygous |
| | | | | | marker |
| D18S548 | 8351 | 8881 | 1.063 | 2 | Normal |
| D21S11 | 33266 | 26998 | 1.232 | 2 | Normal |
| 21-32s | 17806 | 20721 | 1.164 | 2 | Normal |
| D21S1411 | 48630 | 40279 | 1.207 | 2 | Normal |
| CF*508 | 55976 | (—) | (—) | 1 | Normal |
| CF*508del | (—) | (—) | (—) | 0 | |
| Y-40s | 5800 | (—) | (—) | 1 | Male |
| XHPRT | 36707 | (—) | (—) | 1 | Homozygous |
| | | | | | marker |
| DXS7423 | 41823 | (—) | (—) | 1 | Homozygous |
| | | | | | marker |

Multiplex 3

| | | | | | |
|---|---|---|---|---|---|
| Primer | Peak (i) | Peak (ii) | Area ratio | Labels | Diagnosis |

Sample 1

| | | | | | |
|---|---|---|---|---|---|
| D13S627 | 7251 | (—) | (—) | 1 | Homozygous |
| | | | | | marker |
| D13S317 | 129189 | 101651 | 1.271 | 2 | Normal |
| D13S800 | 15073 | 13942 | 1.081 | 2 | Normal |
| D18S978 | 61487 | (—) | (—) | 1 | Homozygous |
| | | | | | marker |
| D18S391 | 108366 | (—) | (—) | 1 | Homozygous |
| | | | | | marker |
| D21S1446 | 44909 | (—) | (—) | 1 | Homozygous |
| | | | | | marker |
| X-61 | 55693 | 42953 | 1.297 | 2 | Disomic X |

Sample 2

| | | | | | |
|---|---|---|---|---|---|
| D13S627 | 65033 | (—) | (—) | 1 | Homozygous |
| | | | | | marker |
| D13S317 | 105284 | (—) | (—) | 1 | Homozygous |
| | | | | | marker |
| D13S800 | 62558 | 68883 | 1.101 | 2 | Normal |
| D18S978 | 21361 | 25572 | 1.197 | 2 | Normal |
| D18S391 | 48434 | 36583 | 1.324 | 2 | Normal |
| D21S1446 | 62455 | (—) | (—) | 1 | Homozygous |
| | | | | | marker |
| X-61 | 22272 | (—) | (—) | 1 | Homozygous |
| | | | | | marker |

Sample 3

| | | | | | |
|---|---|---|---|---|---|
| D13S627 | 39105 | (—) | (—) | 1 | Homozygous |
| | | | | | marker |
| D13S317 | 82568 | 71938 | 1.148 | 2 | Normal |
| D13S800 | 83929 | 68027 | 1.234 | 2 | Normal |
| D18S978 | 35035 | 32673 | 1.072 | 2 | Normal |
| D18S391 | 75961 | 54940 | 1.383 | 2 | Normal |
| D21S1446 | 48155 | 35258 | 1.366 | 2 | Normal |
| X-61 | 35618 | (—) | (—) | 1 | Homozygous |
| | | | | | marker |

1. A method for detecting aneuploidy of a chromosome, the method comprising the steps of:
(a) simultaneously amplifying a plurality of chromosome-specific short tandem repeat (STR) markers to form an amplification product mixture comprising copies of the STR markers;
(b) separating the amplified chromosome-specific STR markers from the amplified product mixture according to size;
(c) determining the relative concentrations of the amplified products corresponding to the chromosome-specific STR markers, and
(d) correlating the relative concentration of each amplified S7R with the presence or absence of aneuploidy of the chromosome, once consistent results are obtained from at least two markers of the chromosome being assayed.
wherein at least three simultaneous assays are carried out according to steps (a) to (c) prior to step (d), in which a peak value ratio of amplification product of a STR marker of 1:1 to 1.4:1 is diagnostic of a normal complement of chromosomes and a peak value ratio of 1.6:1 or above is diagnostic of di-allelic trisomy.

2. A method as claimed in claim 1, in which the aneuploidy is trisomy.

3. A method as claimed in claim 2, in which the trisomy is trisomy 21, trisomy 18, trisomy 13, or trisomy X.

4. A method as claimed in claim 1, in which the aneuploidy is monosomy.

5. A method as claimed in claim 4, in which the monosomy is Turner Syndrome.

6. A method as claimed in any one claims 1 to 5, in which the amplification of the STR markers is by means of the polymerase chain reaction.

7. A method as claimed in any one of claims 1 to 6, in which the reaction product of the amplification reaction is labelled with a fluorescent label.

8. A method as claimed in any one of claims 1 to 7, in which the chromosome to be assayed is obtained from a source of fetal cellular material.

9. A method as claimed in claim 8, in which the source of fetal cellular material is amniotic fluid.

10. A method as claimed in claim 9, in which the source of fetal cellular material is chorionic villus.

11. A method as claimed in any one claims 1 to 7, in which the chromosome to be assayed is obtained from a source of fetal blood

12. A method as claimed in any one claims 1 to 7, in which the chromosome to be assayed is analysed with respect to sample of feral DNA obtained from maternal serum or fetal tissue

13. A method as claimed in any one of claims 1 to 5, in which the at least three simultaneous assays each comprise independently at least six STR markers, and in which a peak value ratio of amplification product of a STR marker of 1:1 to 1.4:1 is diagnostic of a normal complement of chromosomes and a peak value ratio of 1.6:1 or above is diagnostic of di-allelic trisomy.

14. A method as claimed in any one of claims 1 to 13, in which a further amplification assay is performed using additional quantitative markers.

15. A method as claimed in any preceding claim, in which die STR markers comprise D13S257, DXS981, D13S631, D21S1414, DXS6785, D18S536, D13S258, D18S535, D21S1411, D18S548, 21-32S (informal designation), D21S11, DXS7423, HPRT (tor XHPRT), D21S1446, D18S391, X-61 (informal designation), D18S978. D13S317, D13S627, D13S800, or D18S1002, in which the forward primers represented by 21-32S (informal designation) and X-61 (informal designation) are CTC CAG CCT GGG TGA CAA G and ATT GAA GAA GGC ACC TIT CAG C respectively.

16. A method as claimed in any preceding claim, in which a further marker is assayed to detect the presence or absence of a genetic disease.

17. A method as claimed in claim 16, in which the genetic disease is cystic fibrosis.

18. A method as claimed in claim 17, in which the marker used to assay for the presence or absence of cystic fibrosis is CF508 (informal designation), in which the forward primer represented by CF508 (informal designation) is ATG TIT CCT GGA TTA TGC CTG GCA C.

19. A kit of parts comprising at least three multiplexes of labelled primers for the STR markers to be amplified in a method according to claim 1.

20. The use of STR marker 21-32S (informal designation) as a marker for the diagnosis of aneuploidy of a chromosome in a method as defined in claim 1, in which the forward primer represented by 21-32S (informal designation) is CTC CAG CCT GGG TGA CAA G.

21. The use of STR marker Y-40S (informal designation) as a marker for the diagnosis of the sex of an individual in a method as defined in claim 1, in which the forward primer represented by Y405 (informal designation) is GAA TAT TCC CGC TCT CCG GA.
